(19) United States
(12) Patent Application Publication
(10) Pub. No.: US 2021/0023034 A1
(43) Pub. Date: Jan. 28, 2021

(54) PHENYLALANINE DERIVATIVES FOR USE IN THE TREATMENT OF CANCERS

(71) Applicants: UNIVERSITE PARIS EST CRETEIL VAL DE MARNE, CRETEIL (FR); INSTITUT NATIONAL DE LA SANTE ET DE LA RECHERCHE MEDICALE, PARIS (FR); CENTRE NATIONAL DE LA RECHERCHE SCIENTIFIQUE, PARIS (FR); ASSISTANCE PUBLIQUE - HÔPITAUX DE PARIS, PARIS (FR)

(72) Inventors: MARC PRESSET, PARIS (FR); FLAVIA CASTELLANO, ANTONY (FR); VALÉRIE MOLINIER FRENKEL, SAINT-MAUR-DES-FOSSES (FR); ERWAN LE GALL, SAVIGNY SUR ORGE (FR)

(21) Appl. No.: 17/042,218

(22) PCT Filed: Mar. 29, 2019
(86) PCT No.: PCT/EP2019/058077

§ 371 Date: Sep. 28, 2020

(30) Foreign Application Priority Data
Mar. 29, 2018 (EP) 18305358.6

(51) Int. Cl. A61K 31/198; A61K 31/495; A61K 31/4453; A61K 31/5375; A61K 31/404

(52) U.S. Cl. CPC A61K 31/198; A61K 31/495; A61K 31/404; A61K 31/5375; A61K 31/4453

(57) ABSTRACT

The present invention relates to the field of medicine, in particular of oncology. Especially, it provides new compounds useful in the treatment of various cancers. The present invention relates more particularly to methods for the treatment of cancer with IL4I1 expressing cells.

### FIELD OF THE INVENTION

The present invention relates to the field of medicine, in particular of oncology. Especially, it provides new compounds useful in the treatment of various cancers. The present invention relates more particularly to methods for the treatment of cancer with IL4I1 expressing cells.

### BACKGROUND OF THE INVENTION

L-amino acid oxidases (LAAOs) are flavin adenine dinucleotide-dependent enzymes present in all major kingdom of life, from bacteria to mammals. They participate in defense mechanisms by limiting the growth of most bacteria and parasites. A few mammalian LAAOs have been described, of which the enzyme “interleukin-4 induced gene 1” (IL4I1) is the best characterized. The secreted IL4I1 (interleukin-4-induced gene 1) enzyme catabolizes L-phenylalanine and to a lesser extent arginine to generate hydrogen peroxide (H₂O₂), ammonia (NH3) and the corresponding a-keto acid. In cancer patients, IL4I1 is expressed either by tumor cells themselves (e.g., some B cell lymphoma subsets, mesothelioma or ovarian cancer) or by tumor-associated macrophages (TAM) or dendritic cells (DC). It is a secreted enzyme physiologically produced by antigen presenting cells (APC) of the myeloid and B cell lineages and T helper type (Th) 17 cells. Important roles of IL4I1 in the fine control of the adaptive immune response in mice and humans have emerged during the last few years. Indeed, IL4I1 inhibits T cell proliferation and cytokine production and facilitates naïve CD4+ T-cell differentiation into regulatory T cells in vitro by limiting the capacity of T lymphocytes to respond to clonal receptor stimulation. It may also play a role in controlling the germinal center reaction for antibody production and limiting Th1 and Th17 responses. IL4I1 is expressed in tumor-associated macrophages of most human cancers and in some tumor cell types. Such expression, associated with its capacity to facilitate tumor growth by inhibiting the anti-tumor T-cell response, makes IL4I1 a new potential druggable target in the field of immunomodulation in cancer.

In that context, it was disclosed some compounds with IL4I1 inhibitory activity in WO2010/066858. However, the specifically disclosed compounds were found either toxic or with unsatisfactory IL4I1 inhibitory activity. The disclosed compounds had Ki in the mM range, lacking thus efficacy, and rapidly induced complete cell death of a T cell line (Jurkat cells).

Therefore, there is a need for more effective treatment with strong IL4I1 inhibitory activity compounds and for the introduction of new agents in clinical trials.

An object of the invention is thus to find new inhibitors of IL4I1 for use in methods of treatment of cancer.

### SUMMARY OF THE INVENTION

The invention relates to the inhibition of IL4I1 (Interleukin 4 Induced gene 1), which has been found to be expressed in a large set of human cancers. The present invention thus provides a compound for a use in the treatment of cancer, wherein the compound of the inventions is of formula (I), as detailed below. More specifically, the cancer to be treated displays IL4I1-expressing cells.

The invention also relates to a method for treating cancer, more specifically cancer displaying IL4I1-expressing cells, comprising the step of administering to a subject in need thereof a therapeutically effective amount of at least one compound of the invention, which is an inhibitor of IL4I1.

In another aspect, the invention relates to a method for the treatment of cancer, comprising the step of detecting the presence of IL4I1 in a sample obtained from said subject, and then administering to the subject with IL4I1 presenting cells a therapeutically effective amount of at least one compound of the invention.

Accordingly, the compound used according to the invention is with the following formula (I):

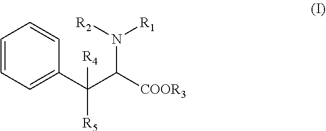

### Wherein:

R1 is a (C₁-C₈)alkyl or (C₆-C₁₈)aryl group, or an heteroaryl group
R2 is an hydrogen atom, a (C₁-C₈)alkyl group, a SO₂R′ group, R′ being an hydrogen atom, an hydroxy group, a (C₁-C₄) alkyl group, or a (C₁-C₄) alkoxy group, or is linked to the phenyl group to form an indoline ring,
alternatively R1 and R2 form together a ring with the nitrogen atom to which they are attached, said ring being optionally substituted with at least one halogen atom or a (C₁-C₈)alkyl group,
R3 is an hydrogen atom, a (C₁-C₈)alkyl group, or a (C₆-C₁₈)aryl(C₁-C₄)alkyl group,
R4 and R5 represent independently an hydrogen atom or a (C₁-C₈)alkyl group,
wherein the phenyl group of formula (I) is optionally substituted with one to five substituents which are independently selected in the group consisting of an halogen atom, a cyano group, a (C₁-C₈)alkyl group,
one of its pharmaceutically acceptable salt, a stereoisomer (diastereoisomer, enantiomer), a racemic mixture, geometric isomers, or a mixture thereof.

The present disclosure also relates to pharmaceutical compositions containing the compounds described therein and more specifically to their use for the treatment of cancers.

### BRIEF DESCRIPTION OF THE DRAWINGS

FIG. 1. IL4I1 Inhibition curves of compounds of the invention: (A) compound 2, (B) compound 7, (C) compound 13, (D) compound 15, and (E) compound 16.

FIG. 2. Effect of the selected compounds (compounds: Cp 2, 7, 13, and 15) on Jurkat-cell growth. *** p<0,001, **** p<0,0001 Two-way ANOVA test. (A) Jurkat cells were seeded at 0.1×10⁶/mL in a 24-well plate and an aliquot mixed with Trypan blue and counted at days 1, 2, and 3. Data are the average±SD of three independent experiments. (B) Jurkat cells were seeded at 0.1×10⁶/mL in a 96-well plate and incubated at 37° C. in 5% CO₂ for the times indicated in the figure. At each time point, cells were centrifuged, the medium removed, and the CellTiter 96® Non-Radioactive Cell Proliferation Assay (Promega) performed. Data are reported as the percentage of proliferation relative to that of the control samples (treated with DMSO) and are the average±SD of three independent experiments. * p<0.01, unpaired t-test.

FIG. 3. Effect of the selected compounds on apoptosis. (A) Activated caspase-3 staining of Jurkat cells incubated with 100 μM of several of the tested molecules. Data are shown as the percentage of Jurkat cells showing activated caspase staining. (B) PBMC. Cells at 0.5×10⁶/mL were seeded in 24-well plates with or without the compounds at 100 μM or 10 μM etoposide (VP16), as a positive control for Jurkat cells apoptosis, and incubated at 37° C. for 24 h. Twenty-four hours after treatment, apoptotic Caspase-3 positive cells were evaluated by FACS. Data are expressed as the percentage of Caspase-3 positive cells±SD from three independent experiments. * p<0.01, ** p<0.001, unpaired t-test.

FIG. 4. Effect of the selected compounds on cell proliferation. Cell proliferation dye fluor670-labeled PBMCs were stimulated to proliferate in 96-well anti-CD3 coated plates and analyzed at days 3 and 6 by FACS gating on the lymphocytes population. (A) A typical FACS analysis at day 3 is shown. (B) The division (left panel) and proliferation indices (right panel) were calculated using FlowJo. Data are representative of three independent experiments.

FIG. 5. A. Representative T cell proliferation in the presence of THP1 control cells (treated with DMSO, gray graph), control THP1-IL4I1 (treated with DMSO, red graph), and Cp2 treated THP1-IL4I1 cells (400 μM, blue graph). B. Proliferation and expansion index of the co-cultures were calculated using the FlowJo proliferation platform. A comparison of three different donors tested in three independent experiments was performed using a paired t-test, (****p<0.0001; ** p<0,001; * p<0,01).

### DETAILED DESCRIPTION

Accordingly, and in a first aspect of the invention, it is herein disclosed a compound of general formula (I) as defined above for use in the treatment of cancer.

According to the invention, the compound in the present invention has the following formula (I):

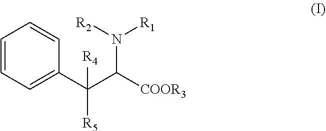

### Wherein:

R1 is a (C₁-C₈)alkyl, (C₆-C₁₈)aryl group, or an heteroaryl group,
R2 is an hydrogen atom, a (C₁-C₈)alkyl group, a SO₂R′ group, R′ being an hydrogen atom, an hydroxy group, a (C₁-C₄) alkyl group, or a (C₁-C₄) alkoxy group, or is linked to the phenyl group to form an indoline ring,
alternatively R1 and R2 form together a ring with the nitrogen atom to which they are attached, said ring being optionally substituted with at least one halogen atom or a (C₁-C₈)alkyl group,
R3 is an hydrogen atom, a (C₁-C₈)alkyl group, or a (C₆-C₁₈)aryl(C₁-C₄)alkyl group,
R4 and R5 represent independently an hydrogen atom or a (C₁-C₈)alkyl group,
wherein the phenyl group depicted in formula (I) is optionally substituted with one to five substituents which are independently selected in the group consisting of an halogen atom, a cyano group, a (C₁-C₈)alkyl group,
one of its pharmaceutically acceptable salt, a stereoisomer (diastereoisomer, enantiomer), a racemic mixture, geometric isomers, or a mixture thereof.

More specifically, the compound in the present invention has the following formula (I):

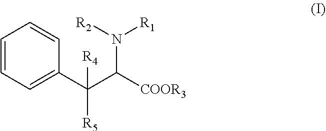

### Wherein:

R1 is a (C₁-C₈)alkyl or (C₆-C₁₈)aryl group,
R2 is an hydrogen atom, a (C₁-C₈)alkyl group or is linked to the phenyl group to form an indoline ring,
alternatively R1 and R2 form together a ring with the nitrogen atom to which they are attached, said ring being optionally substituted with at least one halogen atom or a (C₁-C₈)alkyl group,
R3 is an hydrogen atom, a (C₁-C₈)alkyl group, or a (C₆-C₁₈)aryl(C₁-C₄)alkyl group,
R4 and R5 represent independently an hydrogen atom or a (C₁-C₈)alkyl group,
wherein the phenyl group depicted in formula (I) is optionally substituted with one to five substituents which are independently selected in the group consisting of an halogen atom, a cyano group, a (C₁-C₈)alkyl group,
one of its pharmaceutically acceptable salt, a stereoisomer (diastereoisomer, enantiomer), a racemic mixture, geometric isomers, or a mixture thereof.

According to the invention, the term “(C₁-C₁₀)alkyl” designates a saturated or unsaturated hydrocarbonated group, linear, branched or cyclic, having from 1 to 10, preferably from 1 to 8, from 1 to 6 or from 1 to 4, carbon atoms. Among the saturated alkyl group, one can cite methyl, ethyl, propyl, isopropyl, cyclopropyl, butyl, isobutyl, tert-butyl, cyclobutyl, pentyl, cyclopentyl, neopentyl, n-hexyl. The unsaturated alkyl group can be an alkenyl group or an alkynyl group. The term “alkenyl” refers to an unsaturated, linear, branched or cyclic aliphatic group comprising at least one carbon-carbon double bound. The term “(C₂-C₆)alkenyl includes, but is not limited to, vinyl, —CH₂CH═CH₂, —CH═CH(CH₃), —CH═C(CH₃)₂, —C(CH₃)═CH₂, —C(CH₃)═CH(CH₃), C(CH₂CH₃)═CH₂, cyclohexenyl, cyclopentenyl, cyclohexadienyl, butadienyl, pentadienyl, and hexadienyl, among others. The term “alkynyl” refers to an unsaturated, linear branched or cyclic aliphatic group comprising at least one carbon-carbon triple bound. The term “(C₂-C₆)alkynyl group includes, but is not limited to, 1-ethynyl, 1-propynyl, 2-propynyl, 1-butynyl, 2-butynyl, 1-pentynyl or 2-pentynyl radical, among others.

The alkyl group can be substituted by at least one halogen atom. The alkyl group can be perhalogenated, and in that context the alkyl group being substituted with one or more halogen atoms is more preferably CF₃.

The term “alkoxy group” denotes an alkyl group as defined herein linked to the rest of the compound by an oxygen atom.

The term “aryl” corresponds to a mono- or bi-cyclic aromatic hydrocarbons having from 6 to 18 carbon atoms. For instance, the term “aryl” includes phenyl or naphthyl. In a preferred embodiment, the aryl is a phenyl. The aryl group can be substituted or not. When substituted, the aryl group is preferably substituted by at least one halogen atom, hydroxy group, alkyl group, alkoxy group, aryl group, aryloxy group, acid group (—COOH), cyano, ester group (—COOR, where R is an alkyl as defined herein, preferably a saturated alkyl group) or a mixture thereof, said substituent(s), such as the alkyl group, can also be substituted by at least one halogen atom, hydroxy group, alkoxy group, aryl group, aryloxy group, acid group (—COOH), cyano, ester group (—COOR, where R is an alkyl as defined herein.

The term “aryloxy” corresponds to an aryl group as defined herein linked to the rest of the compound by an oxygen atom.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl. The heteroaryl group can be substituted or not. When substituted, the heteroaryl group is preferably substituted by at least one halogen atom, hydroxy group, alkyl group, alkoxy group, aryl group, aryloxy group, acid group (—COOH), cyano, ester group (—COOR, where R is an alkyl as defined herein, preferably a saturated alkyl group) or a mixture thereof, said substituent(s), such as the alkyl group, can also be substituted by at least one halogen atom, hydroxy group, alkoxy group, aryl group, aryloxy group, acid group (—COOH), cyano, ester group (—COOR, where R is an alkyl as defined herein.

The term arylalkyl refers to an aryl group linked to the rest of the compound by an alkyl group. The aryl and alkyl groups correspond to the definitions given above. Among the arylalkyl group, the benzyl and phenethyl groups are particularly preferred. More specifically, the arylalkyl is a benzyl group.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

The pharmaceutically acceptable salts include salts of a compound of the invention that possess the desired biological activity. The pharmaceutically acceptable salts comprise salts of acid or basic groups present in the compounds specified, inorganic acids as well as organic acids. Representative examples of suitable inorganic acids include hydrochloric, hydrobromic, hydroiodic, nitric, phosphoric, sulfuric and the like. Representative examples of suitable organic acids include formic, acetic, trichloroacetic, trifluoroacetic, propionic, benzoic, cinnamic, citric, fumaric, maleic, methanesulfonic, glyoxylic and the like. The acids can react with the nitrogen atom present in compound of formula (I) to prepare for instance hydrochloride, sulfate, or triflate salt of a compound of formula (I). Suitable basic salts comprise, but are not limited to, salts of aluminium, calcium, lithium, magnesium, potassium, sodium, zinc, and diethanolamine. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of triflate, sulfate, maleate, chlorhydrate, bromhydrate, and methanesulfonate.

The compounds discussed herein also encompass their stereoisomers (diastereoisomers, enantiomers), pure or mixed, racemic mixtures, geometrical isomers, tautomers, salts, hydrates, solvates, solid forms as well as their mixtures. Since the compounds are derivatives of an aminoacid which is the IL4I1 substrate, i.e. phenylalanine, and every amino acid (except glycine) can occur in two isomeric forms, because of the possibility of forming two different enantiomers (stereoisomers) around the central carbon atom linked to the nitrogen atom: L- and D-forms, the compounds of the invention can be in one of both forms or a mixture thereof. In a particular embodiment, the compounds of formula (I) are preferably in the L-form, which is represented as follows:

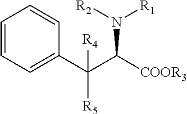

Some compounds according to the invention and their salts could be stable in several solid forms. The present invention includes all the solid forms of the compounds according to the invention which includes amorphous, polymorphous, mono- and polycrystalline forms. The compounds according to the invention can exist in non-solvated or solvated form, for example with pharmaceutically acceptable solvents such as water (hydrates) or ethanol.

According to a particular embodiment, the phenyl group of formula (I) is substituted with one or two substituents, such substituents can be independently selected in the group consisting of chlorine, a (C₁-C₄)alkyl, preferably methyl, cyano or CF₃ group.

When the compound of formula (I) presents R1 which is an aryl group, said aryl, and preferably phenyl, is unsubstituted or substituted by at least one halogen atom, hydroxy group, alkyl group, alkoxy group, aryl group, aryloxy group, acid group (—COOH), cyano, ester group (—COOR, where R is an alkyl as defined herein, preferably a saturated alkyl group) or a mixture thereof, said substituent(s), such as the alkyl group, can also be substituted by at least one halogen atom, hydroxy group, alkoxy group, aryl group, aryloxy group, acid group (—COOH), cyano, and ester group (—COOR, where R is an alkyl as defined herein). According to a more particular embodiment, R1 is a phenyl group substituted by at least one alkyl group, optionally substituted by at least one hydroxy group, acid group (—COOH), ester group (—COOR, where R is an alkyl as defined herein, preferably a saturated alkyl group) or a mixture thereof. More preferably, R1 is a phenyl group substituted by an alkyl group, such as methyl group, substituted by an hydroxy group, and acid group (—COOH) or an ester group (—COOR, where R is an alkyl as defined herein).

According to a particular embodiment, the compound of formula (I) meets at least one of the following features:
R1 is a (C₁-C₄) alkyl group or a phenyl group, optionally substituted as defined above, and R2 is a hydrogen atom or a (C₁-C₄) (preferably (C₂-C₄)) alkyl group,
R1 and R2 form together a ring with the nitrogen atom to which they are attached, wherein said ring is selected in the group consisting of piperazine, morpholine, thiomorpholine and piperidine groups, said ring being optionally substituted as defined in claim 1,
the phenyl group is unsubstituted or substituted with one to five substituents which are independently selected in the group consisting of chlorine, a (C₁-C₄)alkyl, preferably methyl, cyano or CF₃ group,
R3 is an hydrogen atom, a methyl, ethyl, propyl (more preferably n-propyl) or benzyl group,
R4 and R5 represent both hydrogen atoms,
   or a pharmaceutically acceptable salt, a stereoisomer (diastereoisomer, enantiomer), a racemic mixture, geometric isomers, or a mixture thereof.

More particularly, when R1 is a (C₁-C₄) alkyl group, R1 can be a methyl, an ethyl or a propyl (including n-propyl or isopropyl), or a butyl (including n-butyl, sec-butyl, isobutyl or tert-butyl) group.

According to specific embodiments, R1 is more particularly an ethyl or a propyl (including n-propyl or isopropyl), or an alkenyl group, such as a —CH₂CH═CH₂ group. According to another specific embodiment, R1 is not tert-Butyl nor methyl, when R2 is a hydrogen atom. According to other specific embodiments, R1 is a phenyl group, optionally substituted by one or more groups selected from an halogen atom, an hydroxy group, an alkyl group, an alkoxy group, an aryl group, an aryloxy group, an acid group (—COOH), a cyano group, an ester group (—COOR, where R is an alkyl as defined herein, preferably a saturated alkyl group) and a mixture thereof, said substituent(s), such as the alkyl group, can also be substituted by at least one halogen atom, hydroxy group, alkoxy group, aryl group, aryloxy group, acid group (—COOH), cyano, ester group, and R2 is an hydrogen atom or an (C₁-C₄) alkyl, such as methyl.

More specifically, the compound for use according to the invention is selected from the group consisting of:

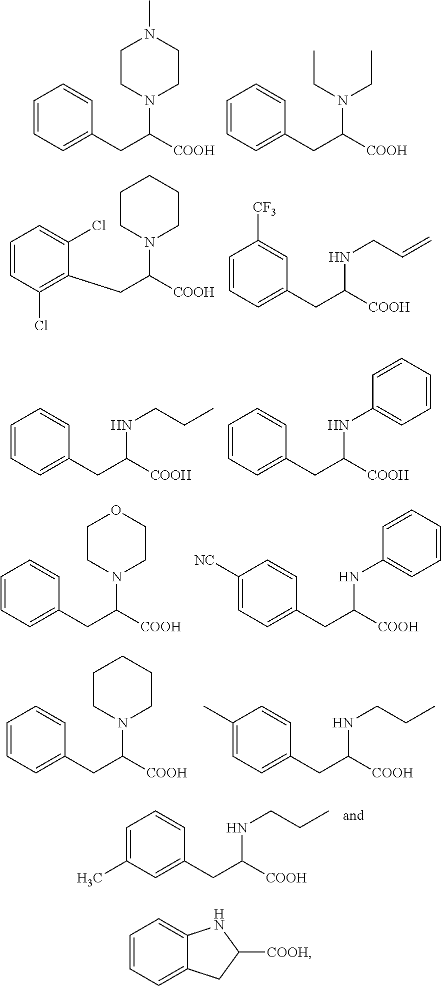

an ester or a salt thereof, preferably the ester is a methyl or ethyl ester of the acid group(s).

According to a more specific embodiment, the compound for use according to the invention is selected in the group consisting of:

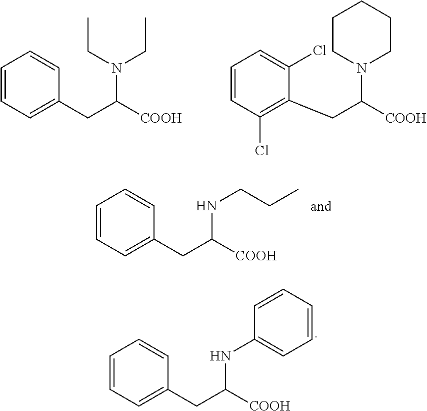

an ester or a salt thereof, preferably the ester is a methyl or ethyl ester of the acid group.

Still more preferably, the compound for use according to the present invention is selected from the group consisting of:

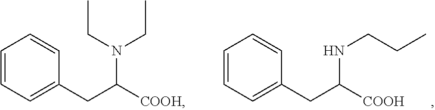 [SMILES: CCN(CC)C(Cc1ccccc1)C(=O)O ; CCCNC(Cc1ccccc1)C(=O)O]

an ester or a salt thereof, preferably the ester is a methyl or ethyl ester of the acid group.

According to another specific embodiment, the compound for use according to the invention is as follows:

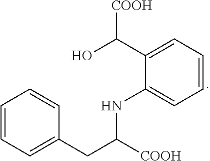 [SMILES: O=C(O)C(Cc1ccccc1)Nc1ccccc1C(O)C(=O)O]

an ester or a salt thereof, preferably the ester is a methyl or ethyl ester of the acid group.

As mentioned above, R3 is an hydrogen atom, a (C₁-C₈)alkyl group, or a (C₆-C₁₈)aryl(C₁-C₄)alkyl group in the compound of formula (I). When R3 represents a (C₁-C₈)alkyl group, it is more particularly a methyl, an ethyl, a propyl (more specifically n-propyl group), or a n-butyl or tertiobutyl group. It is more specifically a methyl or an ethyl group to form an ester on the acid group of the compound of the invention. When R3 is a (C₆-C₁₈)aryl(C₁-C₄)alkyl group, R3 is more particularly a benzyl group.

The compounds according to the present invention may be prepared by various methods known to those skilled in the art. More specifically, the compounds for use according to the invention are prepared as detailed in J. Org. Chem. 2010, 75, 2645-2650 by C. Haurena, E. Le Gall, S. Sengmany, T. Martens and M. Troupel.

According to a particular embodiment, the compound of the invention is for use as a medicament. The present invention also provides a pharmaceutical composition comprising at least one compound as defined above in a pharmaceutically acceptable support. The compound or the pharmaceutical composition of the invention is more particularly for use in the treatment of cancer.

The terms “cancer”, “cancerous”, or “malignant” refer to or describe the physiological condition in subjects that is typically characterized by unregulated cell growth. Said terms refer more specifically according to the invention to cancer displaying IL4I1-expressing cells. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, lung cancer, small-cell lung cancer, non-small cell lung cancer, head or neck cancer, gastrointestinal cancer, renal cancer, ovarian cancer, liver cancer, colorectal cancer, endometrial cancer, kidney cancer, prostate cancer, thyroid cancer, neuroblastoma, osteosarcoma, pancreatic cancer, glioma, glioblastoma multiform, cervical cancer, stomach cancer, bladder cancer, hepatoma, breast cancer, oesophagal cancer, colon carcinoma, and head and neck cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis.

The compound of the invention is particularly for use in the treatment of B cell lymphoid malignancies, such as follicular lymphoma, Hodgkin lymphoma, primary mediastinal B cell lymphoma, diffuse large B cell lymphoma, marginal zone lymphoma and chronic lymphoid leukaemia, ovarian carcinomas, mesotheliomas, colon carcinomas, breast carcinomas, melanomas, glioblastomas and lung carcinomas, more specifically displaying IL4I1-expressing cells.

According to a further specific embodiment, the compound of the invention is for use in the treatment of cancer selected from the group consisting of Primary Mediastinal large B-cell Lymphoma, classical Hodgkin lymphomas, Nodular lymphocyte predominant Hodgkin's lymphoma, non-mediastinal Diffuse Large B-Cell Lymphoma and Small Lymphocytic Lymphoma/Chronic Lymphocytic Leukemia, preferably displaying IL4I1-expressing cells.

As mentioned before, the compounds for use according to the invention are inhibitors of the enzymatic activity of IL4I1, preferably with a Ki lower or equal to 0.2 mM, and more preferably lower or equal to 30 μM.

The compounds are more particularly for the treatment of cancer displaying IL4I1-expressing cells, more specifically IL4I1-expressing tumor cells and/or IL4I1-expressing tumor infiltrating cells of the microenvironment of the primary tumor or the metastasis in the subject.

According to a particular embodiment, the compound as defined above is for use in the treatment of cancer, wherein said treatment is applied to the subject simultaneously, separately or sequentially with another method for treatment of cancer selected from the group comprising surgery, radiotherapy, chemotherapy, hormonal therapy and immuno-therapy, targeted therapy, cells therapy, gene therapy, cancer vaccines, virotherapy, and any combination thereof.

According to another particular embodiment, the compound as defined above is for use in the treatment of cancer, wherein said treatment is applied to a subject not responsive or no longer responsive to immunotherapy treatment in cancer.

The immunotherapy treatment can for instance target one or more of the following pathways: CD27, CD28, CD40, CD137, GITR, ICOS (inducible costimulator), OX40, LAG3, B7-H3. More specifically, immunotherapy treatment can be with the anti-CTLA-4, anti-PD-1, anti-PDL-1, anti LAG3 and/or anti BTLA antibodies

According to another particular embodiment, the compound as defined above is for use in the treatment of cancer, wherein the subject to be treated has been previously determined as being likely to respond to the treatment of cancer with said compound, by having previously detected the expression of IL4I1 in a cancer sample obtained from said subject.

The subject may be a human being or any animal, preferably a human being or a mammal, including cattle, sheep, horses, dogs, cats, goats etc. Poultry, fish or any other animals for food industry are also encompassed. Preferably the subject is a human patient, whatever his/her age or sex. New-borns, infants, children are included as well.

Typically, before applying a method of treatment according to the present invention to a subject suffering from cancer, a diagnostic test may be performed in order to determine whether the cancer displays IL4I1-expressing cells. By performing such a pre-treatment diagnostic test, it is possible to determine whether a subject would be responsive to a method of treatment according to the invention.

Accordingly, the invention also concerns a method for determining the responsiveness of a subject suffering from cancer to an inhibitor of IL4I1 as defined previously, comprising the step of detecting the expression of IL4I1 in a cancer sample obtained from said subject. The cancer sample to detect the expression of IL4I1 can be obtained from tissues, organs, cells, plasma, blood, serum or other biological fluids from the subject, depending on the type of previously diagnosed cancers. In case of tumor cancer, the sample can be a tumor biopsy.

It falls within the ability of the skilled artisan to carry out such a diagnostic test, since detection of the expression of IL4I1 in the above-mentioned cancers can be easily carried out by any usual method known by the skilled person. Typically, IL4I1 expression may be measured through the detection of IL4I1-mRNA in the cell lysate, for example by RT-PCR. IL4I1 expression may also be measured by immunohistochemistry performed on the sample.

The expression of IL4I1 may also be detected by immunological techniques such as ELISA and Western Blot on whole blood sample, plasma sample or serum sample. The expression of IL4I1 may further be detected by the measurement of its enzymatic activity since it was found that enzymatic activity of IL4I1 paralleled IL4I1 expression. The enzymatic activity of IL4I1 may be measured by quantification of H₂O₂, phenylpyruvate or NH₃ produced by IL4I1 phenylalanine oxidative deamination. This test can be carried out on a sample obtained from the subject, such as for example any type of tumor biopsy, whole blood sample, plasma sample or serum sample or other biological fluids. The in vivo enzymatic activity of IL4I1 may also indirectly be measured by quantitation of phenylalanine and phenylpyruvate in biological fluids.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of cancer.

In the context of a treatment, the compounds of the invention may be administered to a subject by any suitable route, including oral, topical, sublingual, parenteral (preferably intravenous), transdermal, rectal, etc. For a brief review of present methods for drug delivery, see, Langer, Science 249:1527-1533 (1990), which is incorporated herein by reference.

The present invention also concerns a pharmaceutical composition comprising a compound of formula (I), as described above, and a pharmaceutically acceptable carrier and/or excipient. This particular aspect also concerns the preferred embodiments disclosed above for the compounds of the invention. In a particular embodiment, the pharmaceutical composition comprises a compound according to any of the above embodiments.

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art. The excipient of the composition can be any pharmaceutically acceptable excipient, including specific carriers able to target specific cells, cellular compartments or tissues. As stated earlier, possible pharmaceutical compositions include those suitable for oral, rectal, topical, transdermal, buccal, sublingual, or parenteral (including subcutaneous, intramuscular, intravenous and intradermal) administration. For these formulations, conventional excipients can be used according to techniques well known by those skilled in the art. The compositions for parenteral administration are generally physiologically compatible sterile solutions or suspensions which can optionally be prepared immediately before use from solid or lyophilized form. For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. For transdermal administration, the composition can be formulated into ointment, cream or gel form and appropriate penetrants or detergents could be used to facilitate permeation, such as dimethyl sulfoxide, dimethyl acetamide and dimethylformamide. For transmucosal administration, nasal sprays, rectal or vaginal suppositories can be used. The compound of the invention can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate. In a preferred embodiment, the pharmaceutical composition of the invention is suitable for parenteral administration.

Pharmaceutical composition according to the invention may be formulated to release the active drug substantially immediately upon administration or at any predetermined time or a time period after administration.

By a “therapeutically effective amount” of a compound according to the invention is meant a sufficient amount to treat cancer, at a reasonable benefit/risk ratio applicable to any medical treatment. It will be understood, however, that the total daily usage of the compound of the invention will be decided by the attending physician within the scope of sound medical judgment. The specific therapeutically effective dose level for any particular subject in need thereof will depend upon a variety of factors including the stage of cancer being treated and the activity of the specific inhibitor/cytotoxic agent employed, the age, body weight, general health, sex and diet of the subject, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

In a particular embodiment, the pharmaceutical composition according to the invention comprises 0.001 mg to 1 g of the compound of the invention. Preferably, pharmaceutical composition according to the invention comprises 0.01 mg to 800 mg of the compound of the invention.

Pharmaceutical compositions according to the invention can comprise one or more compound of the invention in association with pharmaceutically acceptable excipients and/or carriers. These excipients and/or carriers are chosen according to the form of administration as described above.

The below examples illustrate the invention without reducing its scope.

### Examples

### Example 1: Preparation of Compounds of Formula (I)

The following compounds were prepared as detailed in J. Org. Chem. 2010, 75, 2645-2650 by C. Haurena, E. Le Gall, S. Sengmany, T. Martens and M. Troupel.

Deviation from phenylalanine

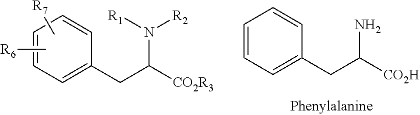

| | | | | | Compound | Compound |
|---|---|---|---|---|---|---|
| R6 = | R7 = | R1 = | R2 = | R3 = | structure | number |
| H | H | CH₂CH₃ | CH₂CH₃ | CH₂CH₃ | 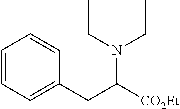 | 13 |
| H | H | CH₂CH₂CH₃ | H | CH₂CH₃ | 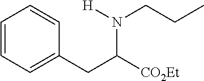 | 2 |
| 3-CF₃ | H | CH₂CHCH₂ | H | CH₂CH₃ | 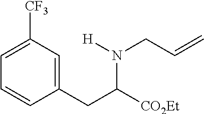 | 16 |
| H | H | C₆H₅ | H | CH₂CH₃ | 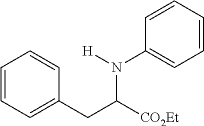 | 15 |
| 4-CN | H | C₆H₅ | H | CH₂CH₃ | 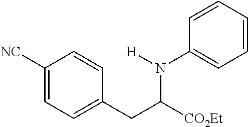 | 17 |

| | | | | | |
|---|---|---|---|---|---|
| H | H | CH₂CH₂CH₂CH₂CH₂ | CH₂CH₃ | 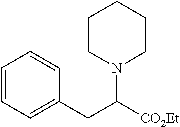 | 11 |
| 2-CI | 6-CI | CH₂CH₂CH₂CH₂CH₂ | CH₂CH₃ | 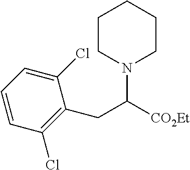 [SMILES: CCOC(=O)C(Cc1c(Cl)cccc1Cl)N1CCCCC1] | 7 |
| H | H | CH₂CH₂N(CH₃)CH₂CH₂ | CH₂CH₃ | 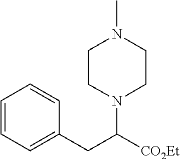 | 1 |
| H | H | CH₂CH₂OCH₂CH₂ | CH₂CH₃ | 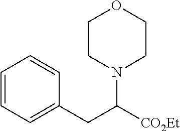 | 10 |
| H | H | See compound structure | CH₂CH₃ | 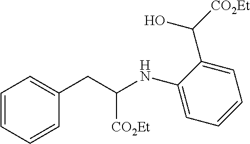 | 16 |

“Et” stand for ethyl

Sulfamic Acid and Sulfamide of Compound 2 (Cp2) were Also Prepared.

Compound sulfamide was prepared as follows. To a solution of Cp2 (235 mg, 1.0 mmol) in CH₂Cl₂ (2 mL) at 0° C. under Ar was added triethylamine (0.3 mL, 2.0 equiv) and mesyl chloride (0.1 mL, 1.1 equiv). The reaction was stirred at 0° C. for 2 h. Then, the reaction mixture was poured into sat. aq. NH₄Cl (10 mL) and extracted twice with CH₂Cl₂ (2×10 mL). The combined organic layers were dried (Na₂SO₄) and evaporated. Purification of the residue by flash chromatography afforded the title compound (176 mg, 56%). 1H NMR (400 MHz, CDCl₃): δ 7.32-7.25 (m, 5H), 4.74 (t, J=7.4 Hz, 1H), 4.19 (q, J=7.0 Hz, 2H), 3.38 (dd, J=14.1, 7.4 Hz, 1H), 3.28-3.20 (m, 1H), 3.13-3.03 (m, 2H), 2.73 (s, 3H), 1.691.58 (m, 2H), 1.26 (t, J=7.0 Hz, 3H), 0.89 (t, J=7.4 Hz, 3H). 13C NMR (400 MHz, CDCl₃): δ 170.8 (C), 137.1 (C), 129.3 (2 CH), 128.6 (2 CH), 127.0 (CH), 61.8 (CH), 61.7 (CH₂), 48.0 (CH₂), 39.9 (CH₃), 37.0 (CH₂), 23.4 (CH₂), 14.1 (CH₃), 11.4 (CH₃).

Compound sulfamic acid was prepared as follows. To a solution of Cp2 (82 mg, 1.0 mmol) in CH₂Cl₂ (2 mL) at 0° C. under Ar was added chlorosulfonic acid (25 μL, 1.1 equiv). The reaction was stirred at 0° C. for 2 h. Then, the reaction mixture was evaporated and Et₂O (10 mL). The resulting mixture was stirred overnight at rt and the title compound was collected by filtration (89 mg, 80%). 1H NMR (400 MHz, CDCl₃): δ 8.96 (s, 1H), 7.29-7.24 (m, 5H), 4.19 (br s, 1H), 4.07 (q, J=7.1 Hz, 2H), 3.63 (dd, J=13.1, 4.6 Hz, 1H), 3.29-3.23 (m, 1H), 3.11 (br s, 1H), 2.98 (br s, 1H), 1.93-1.86 (m, 2H), 1.03 (t, J=7.1 Hz, 3H), 0.93 (t, J=7.3 Hz, 3H). 13C NMR (400 MHz, CDCl₃): δ 168.2 (C), 134.2 (C), 129.5 (2 CH), 128.8 (2 CH), 127.6 (CH), 62.6 (CH), 61.9 (CH₂), 49.4 (CH₂), 36.4 (CH₂), 19.6 (CH₂), 13.8 (CH₃), 11.0 (CH₃).

Compound 16 (Cp16) was prepared as follows. To a solution of Cp15, an alkoxycarbonyl group was added in ortho. This compound is obtained as by-product of Cp15 due to an excess of reactivity of the glyoxylate which is trapped by Friedel-Crafts type reaction in these conditions and is then isolated by separation during silica chromatography.

### Example 2: Biological and Stability Assays

In the following description, all biological experiments for which no detailed protocol is given are performed according to standard protocols

### Materials and Methods

Cells. PBMCs, obtained from healthy donors by cytapheresis, were provided by the Etablissement Francais du sang (EFS) and separated using Unisep tubes filled with lymphocyte separation medium (EuroBio, France). The Jurkat T cell line and human PBMCs were cultivated in RPMI 1640 medium containing 10% fetal calf serum and 50 U/mL penicillin and 50 μg/mL streptomycin.

Compounds. Stock solutions were prepared by suspending the compounds 2, 7, 13, 15 and 16 as detailed above in DMSO at a concentration of 400 mM and stored at −20° C. Working solutions of the compounds were diluted in DMSO and added at a 1:1000 dilution to the enzyme solution.

Evaluation of Ki. Recombinant human IL4I1 (from R&D Systems, Inc) was diluted in PBS at 100 g/mL and pre-incubated for 30 min with decreasing amounts of each inhibitor dissolved in DMSO (50, 25, and 2.5 μM) or DMSO as a control. The activity was tested in the presence of various amounts of substrate (0.25, 0.5, 1, 2, or 2 mM phenylalanine) using the fluorometric IL4I1 assay described by Carbonnelle-Puscian et al. (Leukemia, 2009, volume 23, pages 952-960 (2009). Ki were calculated using Graph Pad Prism.

Cell counts. Jurkat cells were seeded at 0.1×10⁶/mL in a 24-well plate and an aliquot mixed with Trypan blue and counted at days 1, 2, 3, and 6 using disposable Fast-read 10 chambers. Evaluation of metabolic proliferation. Jurkat cells were seeded at 0.1×10⁶/mL in a 96-well plate and incubated at 37° C. in 5% CO₂ for various times. At each time point, cells were centrifuged, the medium removed, and CellTiter 96® Non-Radioactive Cell Proliferation Assay reagent (Promega) added and the cells incubated an additional 4 h before measuring the optical density at 570 nm in a plate reader (BMG Labtech Fluostar Optima).

Evaluation of Apoptosis. Cells were seeded at 0.5×10⁶/mL in 24-well plates, with or without the compounds at 100 μM or 10 μM etoposide (VP16) as a positive control, and incubated at 37° C. for 24 h. Cells were then fixed and permeabilized using cytox/cytoperm solution (BD Bioscience) followed by the addition of anti-activated caspase 3-AlexaFluor 647 (Rabbit anti-active Caspase 3 clone C92-605, BD Pharmingen). Caspase-3 positive cells were evaluated using a Cyan ADP LX7 (Beckman Coulter) and analyzed using Flowjo.

Evaluation of cell proliferation. Jurkat cells and PBMCs were labeled with 2 μM Cell Proliferation Dye (CPD) eFluor670 for 10 min at 37° C. DMSO or the compounds were added to labeled PBMCs or Jurkat cells before putting them in culture at 0.2×10⁶/well. Jurkat cells were cultured under standard conditions, whereas PBMCs were cultured in 96 U-shaped wells pre-coated with a 0.5 μg/mL solution of anti-CD3 (clone OKT3) functional quality antibody (eBioscience). Cells were collected at day 0, 3, and 6 for fluorescence analysis using a Cyan ADP LX7 (Beckman Coulter). Data were analyzed using Flowjo.

Proliferation experiments: Peripheral blood mononuclear cells (PBMC) were obtained by cytapheresis of blood from consenting donors from the Etablissement Francais du sang (EFS) and purified using Unisep separation tubes (Eurobio). PBMC were labeled with 1 μM Cell Proliferation Dye 670 (CPD, Thermo Fisher) for 10 min at 37° C. After the addition of 5× complete medium and several washes in complete medium to remove the excess non-incorporated CPD, 2×10⁵ PBMC were incubated with 0.25×10⁶ THP1 or THP1 cells stably expressing IL4I1 in the presence of increasing concentrations of Cp2-SO₄ (sulfate salt of compound 2) dissolved in DMSO (100, 200, or 400 μM) or the equivalent volume of DMSO in THP1 or THP1-IL4I1 controls. Proliferation was induced by the addition of 10 or 20 ng/mL of anti-CD3 antibodies (clone OKT3). Cells were analyzed by flow cytometry (BD Fortessa X20) at day 5 and proliferation and expansion indexes calculated using FlowJo 10 software.

### Experiments and Results

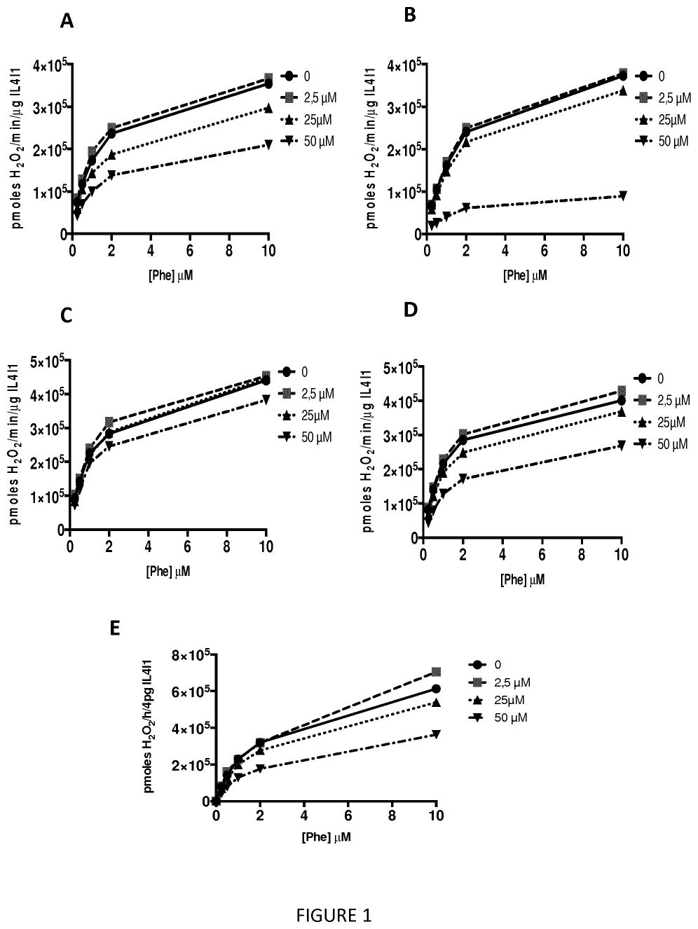

Inhibition curves of compounds 2, 7, 13, 15 and 16 are given in FIG. 1. Recombinant human IL4I1 was diluted in PBS at 100 g/mL and pre-incubated for 30 min with decreasing amounts of each inhibitor dissolved in DMSO (50, 25, or 2.5 μM) or DMSO alone, as a control, and IL4I1 measured. Table 2 below shows the compounds tested and the measured Ki (average of three experiments). All the tested compounds inhibit IL4I1 activity, with the highest Ki for compound 7, followed by 15 and 2. Compound 13 inhibits IL4l1 activity with an almost 10-fold higher Ki.

TABLE 2

| | IL4I1 | |
|---|---|---|
| | Inhibitor | Ki (μM) |
| Cp 2 | Yes | 21.26 |
| Cp 7 | Yes | 9.8 |
| Cp 13 | Yes | 102.41 |
| Cp 15 | Yes | 26.89 |
| Cp 16 | Yes | 25.01 |

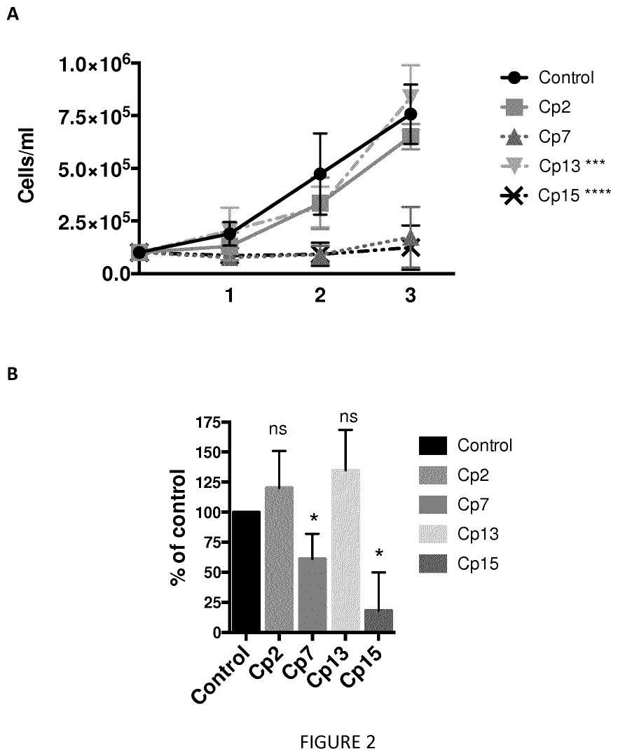

Effects of the selected compounds on Jurkat-cell growth are given in FIG. 2. (A) Jurkat cells were seeded at 0.1×10⁶/mL in a 24-well plate and an aliquot mixed with Trypan blue and counted at days 1, 2, 3, and 6. Data are the average±SD of three independent experiments. (B) Jurkat cells were seeded at 0.1×10⁶/mL in a 96-well plate and incubated at 37° C. in 5% CO2 for the times indicated in FIG. 2. At each time point, cells were centrifuged, the medium removed, and the CellTiter 96® Non-Radioactive Cell Proliferation Assay (Promega) performed. Data are reported as the percentage of proliferation relative to that of the control samples (treated with DMSO) and are the average±SD of three independent experiments. *** p<0,001, **** p<0,0001 Two-way ANOVA test.

Compound 2 and 13 do not affect cell proliferation, measured either by cell counting or metabolic labeling, whereas compound 7 strongly inhibits proliferation. Compound 15 is toxic and no cells remained after six days of culture.

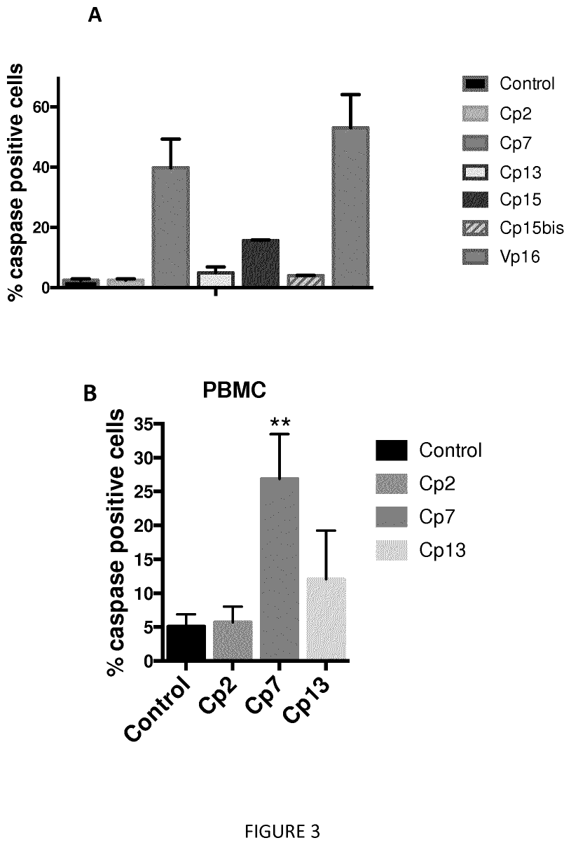

Effects of the selected compounds on apoptosis are given in FIG. 3. (A) Activated caspase-3 staining of Jurkat cells and (B) PBMC. Cells at 0.5×10⁶/mL were seeded in 24-well plates with or without the compounds at 100 μM or 10 μM etoposide (VP16), as a positive control, and incubated at 37° C. for 24 h. Twenty-four hours after treatment, apoptotic Caspase-3 positive cells were evaluated by FACS. Data are expressed as the percentage of Caspase-3 positive cells±SD from three independent experiments.

The apoptosis of both Jurkat cells and PBMCs is induced by compound 7, whereas compound 2 and 13 do not induce significant apoptosis in either case. Compound 16 (also referred as Cp15bis in FIG. 3A) does not induce apoptosis of Jurkat cells.

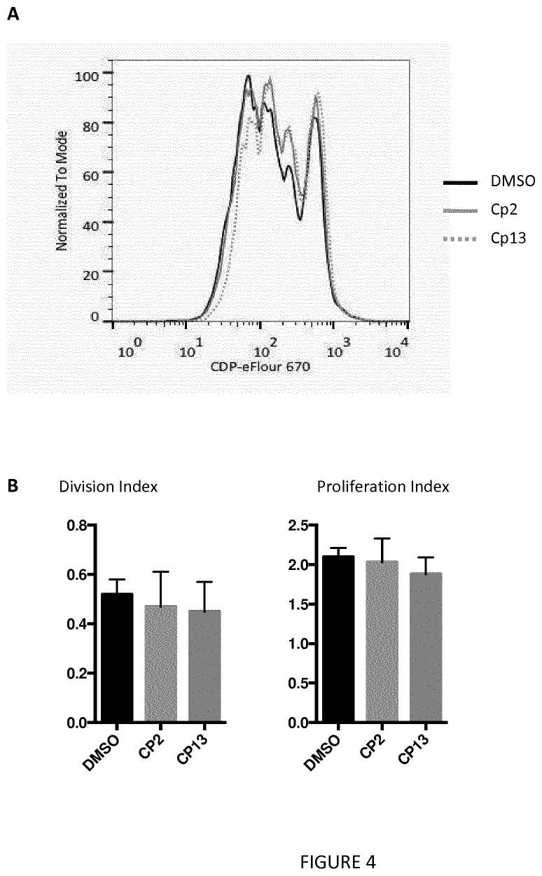

Effects of the selected compounds on cell proliferation are given in FIG. 4. Cell proliferation dye fluor670-labeled PBMCs were stimulated to proliferate in 96-well anti-CD3 coated plates and analyzed at days 3 and 6 by FACS gating on the lymphocytes population. (A) A typical FACS analysis at day 3 is shown. (B) The division (left panel) and proliferation indices (right panel) were calculated using FlowJo. Data are representative of three independent experiments.

The proliferation of Jurkat cells (not shown) and PBMCs is not substantially modified by compound 2 or compound 13. Both the mean number of proliferating cells (proliferation index) and cell divisions of the entire cell population (division index) do not vary markedly in the presence of compound 2 or compound 13, relative to the cells treated with DMSO.

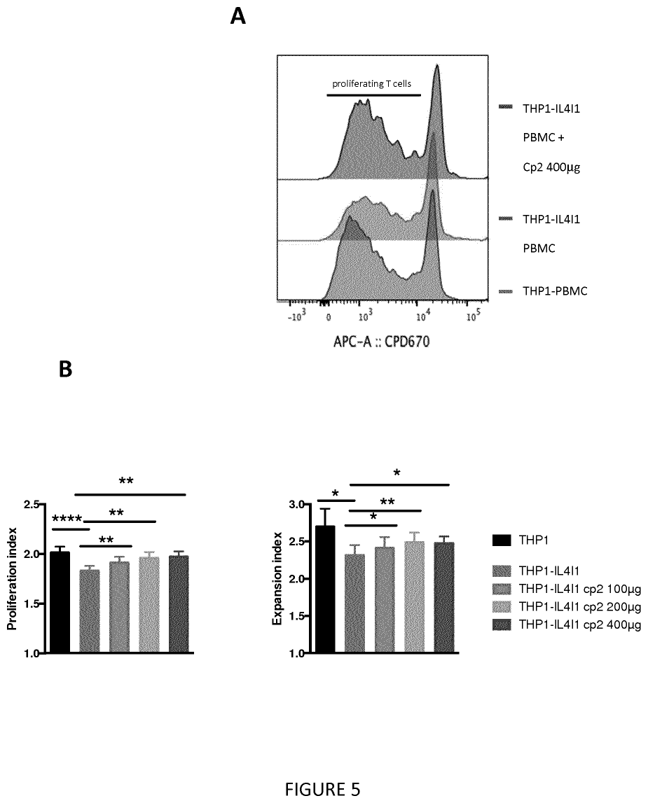

FIG. 5. Cp2-SO₄ (sulfate salt of compound 2) can reverse Il4i1 inhibitory effect. Co-cultures of CPD-labeled PBMC and irradiated (200 Gy) THP1 cells, expressing or not recombinant IL4I1, were stimulated with anti-CD3 antibodies to stimulate T lymphocyte proliferation. The THP1-IL4I1 co-cultures were treated with increasing concentrations of the cp2-SO₄ inhibitor (100, 200, and 400 μM) and the proliferation measured at day 5 by flow cytometry. A. Representative T cell proliferation in the presence of THP1 control cells (treated with DMSO, gray graph), control THP1-IL4I1 (treated with DMSO, red graph), and cp2 treated THP1-IL4I1 cells (400 μM, blue graph). B. Proliferation and expansion index of the co-cultures were calculated using the FlowJo proliferation platform. A comparison of three different donors tested in three independent experiments was performed using a paired t-test, (****p<0.0001; ** p<0,001; * p<0,01).

The proliferation index reveals the mean number of cell divisions done by the cells that have divided in the culture. The expansion index indicates a factor of amplification of the total population. Both indexes are significantly diminished by IL4I1 and brought back to values similar to those of the co-cultures in the presence of THP1 not expressing IL4I1. Thus, the inhibitory effect of IL4I1 on T-lymphocyte proliferation is reversed by Cp2-SO₄ in a dose dependent way.

Table 3 below shows the compounds disclosed in WO2010/06685 tested for IL4I1 inhibitory activity and their measured Ki (average of three experiments). All the tested compounds had a Ki in the range of mM, thus much higher than the measured Ki of compounds 2, 7, 13 and 15.

TABLE 3

| Inhibitor | Ki (mM) |
|---|---|
| L-phenylalanine ethyl ester | 3.1 |
| N-acetyl-phenylalanine | 2.1 |
| 2′-aza-phenylalanine | 2.8 |

1-13. (canceled)

14. A method for the treatment of cancer comprising the step of administering to a subject in need thereof a therapeutically effective amount of at least one compound of formula (I) or a pharmaceutically acceptable salt, a stereoisomer, a racemic mixture, geometric isomer, or a mixture thereof, wherein formula (I) is as follows: 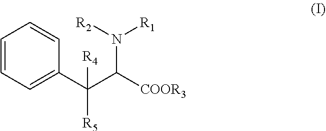
R1 is a (C₁-C₈)alkyl, (C₆-C₁₈)aryl group or heteroaryl group,
R2 is an hydrogen atom, a (C₁-C₈)alkyl group, a SO₂R′ group, R′ being an hydrogen atom, an hydroxy group, a (C₁-C₄) alkyl group, or a (C₁-C₄) alkoxy group, or is linked to the phenyl group to form an indoline ring,
alternatively R1 and R2 form together a ring with the nitrogen atom to which they are attached, said ring being optionally substituted with at least one halogen atom or a (C₁-C₈)alkyl group,
R3 is an hydrogen atom, a (C₁-C₈)alkyl group, or a (C₆-C₁₈)aryl(C₁-C₄)alkyl group,
R4 and R5 represent independently an hydrogen atom or a (C₁-C₈)alkyl group,
wherein the phenyl group of formula (I) is optionally substituted with one to five substituents which are independently selected in the group consisting of an halogen atom, a cyano group, a (C₁-C₈)alkyl group.

15. The method according to claim 14, wherein the compound of formula (I) meets at least one of the following features:
R1 is a (C₁-C₄) alkyl group or a phenyl group, and R2 is an hydrogen atom or a (C₂-C₄) alkyl group,
R1 and R2 form together a ring with the nitrogen atom to which they are attached, wherein said ring is selected in the group consisting of piperazine, morpholine, thiomorpholine and piperidine groups, said ring being optionally substituted with at least one halogen atom or a (C₁-C₈)alkyl group, R3 is an hydrogen atom, a (C₁-C₈)alkyl group, or a (C₆-C₁₈)aryl(C₁-C₄)alkyl group,
the phenyl group is unsubstituted or substituted with one to five substituents which are independently selected in the group consisting of chlorine, a (C₁-C₄)alkyl, cyano or CF₃ group,
R3 is an hydrogen atom, a methyl, ethyl, propyl or benzyl group, and
R4 and R5 represent hydrogen atoms.

16. The method according to claim 14, wherein the compound is of formula (I) where R1 is an aryl group that is unsubstituted or substituted by at least one halogen atom, hydroxy group, alkyl group, alkoxy group, aryl group, aryloxy group, acid group, cyano, ester group or a mixture thereof, said substituent(s) optionally being substituted by at least one halogen atom, hydroxy group, alkoxy group, aryl group, aryloxy group, acid group, cyano, or ester group.

17. The method according to claim 14, wherein the compound is of formula (I) where R1 is a phenyl group, optionally substituted by one or more groups selected from an halogen atom, an hydroxy group, an alkyl group, an alkoxy group, an aryl group, an aryloxy group, an acid group, a cyano group, an ester group and a mixture thereof, said substituent(s) being optionally substituted by at least one halogen atom, hydroxy group, alkoxy group, aryl group, aryloxy group, acid group, cyano, ester group, and R2 is an hydrogen atom or a (C₁-C₄) alkyl.

18. The method according to claim 14, wherein the compound of formula (I) is selected from the group consisting of: 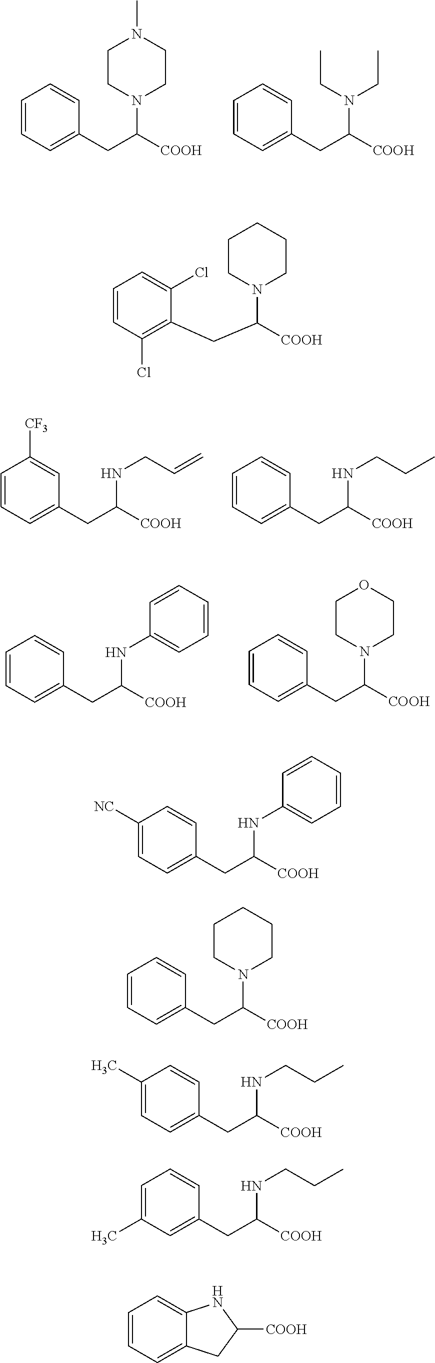
and
an ester or a salt thereof.

19. The method according to claim 14, wherein said compound is selected from the group consisting of: 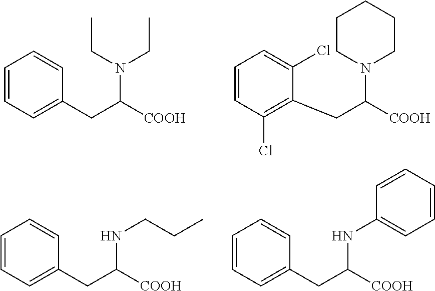
and
an ester or a salt thereof.

20. The method according to claim 14, wherein said compound is: 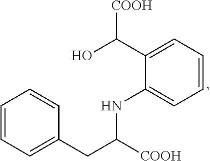 [SMILES: O=C(O)C(Cc1ccccc1)Nc1ccccc1C(O)C(=O)O]
an ester or a salt thereof.

21. The method according to claim 14, wherein said compound is an inhibitor of the enzymatic activity of IL4I1.

22. The method according to claim 14, wherein the cancer to be treated displays IL4I1-expressing cells in the human or animal body.

23. The method according to claim 14, wherein said cancer is selected from the group consisting of of B cell lymphoid malignancies, follicular lymphoma, Hodgkin lymphoma, primary mediastinal B cell lymphoma, diffuse large B cell lymphoma, marginal zone lymphoma, chronic lymphoid leukaemia, ovarian carcinomas, mesotheliomas, colon carcinomas, breast carcinomas, melanomas, glioblastomas and lung carcinomas.

24. The method according to claim 14, wherein said treatment is applied to a subject simultaneously, separately or sequentially with another method for treatment of cancer selected from the group consisting of surgery, radiotherapy, chemotherapy, hormonal therapy and immunotherapy, targeted therapy, cells therapy, gene therapy, cancer vaccines, virotherapy, and a combination thereof.

25. The method according to claim 14, wherein said treatment is applied to a subject not responsive or no longer responsive to immunotherapy treatment in cancer.

26. The method according to claim 14, wherein the subject to be treated has been previously determined as being likely to respond to the treatment of cancer with said compound, by having previously detected the expression of IL4I1 in a cancer sample obtained from said subject.

27. The method according to claim 21, wherein the inhibitor has a Ki lower or equal to 0.2 mM.

28. The method according to claim 14, wherein the immunotherapy treatment is treatment with anti-CTLA-4, anti-PD-1, anti-PDL-1, anti LAG3 or anti BTLA antibodies.
